(19) United States
(12) Patent Application Publication
(10) Pub. No.: US 2025/0032505 A1
(43) Pub. Date: Jan. 30, 2025

(54) Methods And Compositions For Radioprotection

(71) Applicant: Steven BARANOWITZ, Wyncote, PA (US)

(72) Inventor: Steven BARANOWITZ, Wyncote, PA (US)

(21) Appl. No.: 18/562,394

(22) PCT Filed: May 19, 2022
(86) PCT No.: PCT/US2022/029962

§ 371 Date: Nov. 20, 2023

Related U.S. Application Data
(60) Provisional application No. 63/193,146, filed on May 26, 2021.

(51) Int. Cl. A61K 31/555; A61P 43/00

(52) U.S. Cl. CPC A61K 31/555; A61P 43/00

(57) ABSTRACT

Provided herein are compositions and methods for radioprotection. A pharmaceutical composition comprising: a therapeutically effective amount of a bismuth-melanin composite, pharmaceutically acceptable salts thereof, and combinations thereof; and at least one pharmaceutically acceptable excipient and methods for the treatment and/or prevention of acute radiation syndrome (ARS) in a subject in need thereof comprising administering the composition.

### CROSS-REFERENCE TO RELATED APPLICATION

This international application claims benefit of U.S. Ser. No. 63/193,146 filed May 26, 2021, the entirety of which is incorporated herein by reference.

### SPECIFICATION

### Background

This disclosure provides a bismuth-melanin combination which absorbs x-rays (medical quality), and other forms of radiation, at least as well as lead, pharmaceutical compositions comprising bismuth-melanin composites, and radiation shielding comprising bismuth-melanin.

El-Bialy et al, 2019 reported that a Bismuth-Melanin combination was able to absorb gamma rays at least twice as well as lead, and weighed about half as much as lead. (El-Bialy, H. et al. Microbial melanin physiology under stress conditions and gamma radiation protection studies. Radiation Physics and Chemistry. Volume 162, September 2019, pgs. 178-186). The El-Bialy reference discloses new composites to use as shielding materials. These composites are based on fungal melanin's chelating activity with nanoparticles of bismuth, lead, and silver. The mass attenuation coefficient of Melanin-Bismuth composite is nearly double the mass attenuation of lead at energy=0.662 MeV; a higher increase in mass attenuation is observed with higher gamma intensities; 1.17 and 1.33 MeV. El-Bialy Attenuation Measurement Method. The attenuation coefficient of the melanin-heavy metals composites were measured in narrow beam transmission geometry by using a NaI (TI) crystal detector with an energy resolution of 14.0% radio-active sources 60Co (with energies 1.17 and 1.33 MeV) and 137Cs (with energy=0.662 MeV); Incident and transmitted intensities of photons are measured on multichannel analyzer at a fixed preset time for each sample by selecting a narrow region symmetrical with respect to the centroid of the photopeak with counting time in the range of 102-104 min.

TABLE 1
The mass attenuation coefficients of new composites based on microbial melanin
at 0.662, 1.17 and 1.33 MeV energies in comparison with reference materials.

| | | | Mass attenuation coefficient | | |
|---|---|---|---|---|---|
| | | | [symbol] | [symbol] | |
| | Material | Density | 0.662 MeV | 1.17 MeV | 1.33 MeV |
| New composites | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| Control | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| Reference materials | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |

| | | | Mass attenuation coefficient | | |
|---|---|---|---|---|---|
| | | | [symbol] | [symbol] | |
| | Material | Density | 0.662 MeV | 1.17 MeV | 1.33 MeV |
| 1) | Bismuth | 9.78 | 0.1160 | 0.0610 | 0.0580 |
| 2) | M—Bi | 4.51 | 0.2024 | 0.1732 | 0.1570 |
| 3) | Lead | 11.34 | 0.1120 | 0.0617 | 0.0561 |
| 4) | M—Pb | 4.67 | 0.1822 | 0.1469 | 0.1332 |

[symbol]

indicates data missing or illegible when filed

The El-Bialy method for production of new composites involved in 15 gm of bismuth oxide (Bi2O3), lead nitrate [Pb(No3)2] or/and silver nitrate (AgNo3) mixed with 1 g of yeast melanin dissolved in 20 ml of 1N NaOH. The suspensions were vigorously mixed in a water bath at 100° C. for 10 min until complete homogeneity. Then, particles of new composites were dried in an oven at 60° C. and compressed in the form of circular discs using a hydraulic press (1.471 Bar).

The inventor of this disclosure has found that a bismuth-melanin combination could be made which would absorb x-rays (medical quality) at least as well as lead. In addition, because the specific density of the bismuth-melanin combination was about half that of an equivalent volume of lead, it was concluded that the bismuth melanin combination weighed about half that of lead, but absorbed x-rays at least as well as lead, which would have great utility in shielding. Furthermore, since both bismuth and melanin are essentially non-toxic (LD50 of bismuth is about 2000 mg/kg; LD50 of melanin is about >5000 mg/kg), the bismuth-melanin combination can be administered as a medicament to prevent and treat radiation poisoning.

All references cited herein are incorporated herein by reference in their entireties.

### BRIEF SUMMARY

The disclosure provides a pharmaceutical composition comprising: a therapeutically effective amount of a bismuth-melanin composite, pharmaceutically acceptable salts thereof, and combinations thereof, and at least one pharmaceutically acceptable excipient.

The disclosure provides a method for the treatment and/or prevention of acute radiation syndrome (ARS) in a subject in need thereof comprising administering the composition as disclosed herein to the subject, wherein the administration of the composition treats and/or prevents ARS in the subject. The disclosure provides a method wherein the radiation is selected from the group consisting of beta particles, gamma rays, X-rays, infrared, visible, ultraviolet, the remainder of the electromagnetic spectrum, and combinations thereof.

The disclosure provides a kit for the treatment, amelioration or prevention of a condition selected from the group consisting of acute radiation syndrome (ARS), in a patient in need thereof comprising: (a) the pharmaceutical composition as disclosed herein; and (b) at least one blister package; a lidded blister; a blister card or packet; a clamshell; an intravenous (IV) package, IV packette or IV container; a bottle; a metal tube; a laminate tube; a plastic tube; a dispenser; a pressurized container; a barrier container; a package; a tray or a shrink wrap, comprising the pharmaceutical composition of (a) and instructions for use of the pharmaceutical composition.

The disclosure provides a product of manufacture comprising a blister package; a lidded blister; a blister card or packet; a clamshell; an intravenous (IV) package, IV packette or IV container; a bottle; a metal tube; a laminate tube; a plastic tube; a dispenser; a pressurized container; a barrier container; a package; a tray or a shrink wrap comprising the pharmaceutical composition as disclosed herein and instructions for use of the composition.

The disclosure provides a radiation protection material formed using a composition comprising a bismuth-melanin composite; and optionally additional materials. The disclosure provides a radiation protection material as disclosed herein wherein the radiation protection material is manufactured in a form selected from the group consisting of particles, nanoparticles, dust, beads, fibers that are woven, fibers that are non-woven, sheets, films, slabs, plates, bricks, chars, spheres, nodules, balls, graphite-like sheets and shards, liquids, gels, solids, thermoplastic solids, and thermoset solids.

The disclosure provides a process for forming a radiation protection material comprising mixing a bismuth-melanin composite; and at least one additional non-melanin material; and shaping the resultant material into an article. The disclosure provides a process for forming a radiation protection material as disclosed herein wherein the radiation protection material is manufactured in a form selected from the group consisting of particles, nanoparticles, dust, beads, fibers that are woven, fibers that are non-woven, sheets, films, plates, bricks, chars, spheres, nodules, balls, graphite like sheets and shards, liquids, gels, solids, thermoplastic solids, and thermoset solids. The disclosure provides a process for forming a radiation protection material as disclosed herein wherein the radiation protection material absorbs radiation selected from the group consisting of beta particles, gamma rays, X-rays, infrared, visible, ultraviolet, the remainder of the electromagnetic spectrum, and combinations thereof.

The disclosure provides for the use of the compositions of the disclosure for the production of a medicament for preventing and/or treating the indications as set forth herein.

In accordance with a further embodiment, the present disclosure provides a use of the pharmaceutical compositions described above, in an amount effective for use in a medicament, and most preferably for use as a medicament for treating a disease or disorder, for example, as set forth in herein, in a subject.

In accordance with yet another embodiment, the present disclosure provides a use of the pharmaceutical compositions described above, and at least one additional therapeutic agent, in an amount effective for use in a medicament, and most preferably for use as a medicament for treating a disease or disorder associated with disease, for example, as set forth herein, in a subject.

The disclosure provides a method for treating and/or preventing a disease or condition as set forth herein in a patient, wherein said method comprises: selecting a patient in need of treating and/or preventing said disease or condition as set forth herein; administering to the patient a composition of the disclosure in a therapeutically effective amount, thereby treating and/or preventing said disease in said patient.

### BRIEF DESCRIPTION OF SEVERAL VIEWS OF THE DRAWINGS

The invention will be described in conjunction with the following drawings in which like reference numerals designate like elements and wherein:

FIG. 1 is a photograph showing a bismuth-melanin disk made with the methods of the disclosure.

FIG. 2 is a radiograph showing a comparison of a lead rectangle and a bismuth-melanin puck.

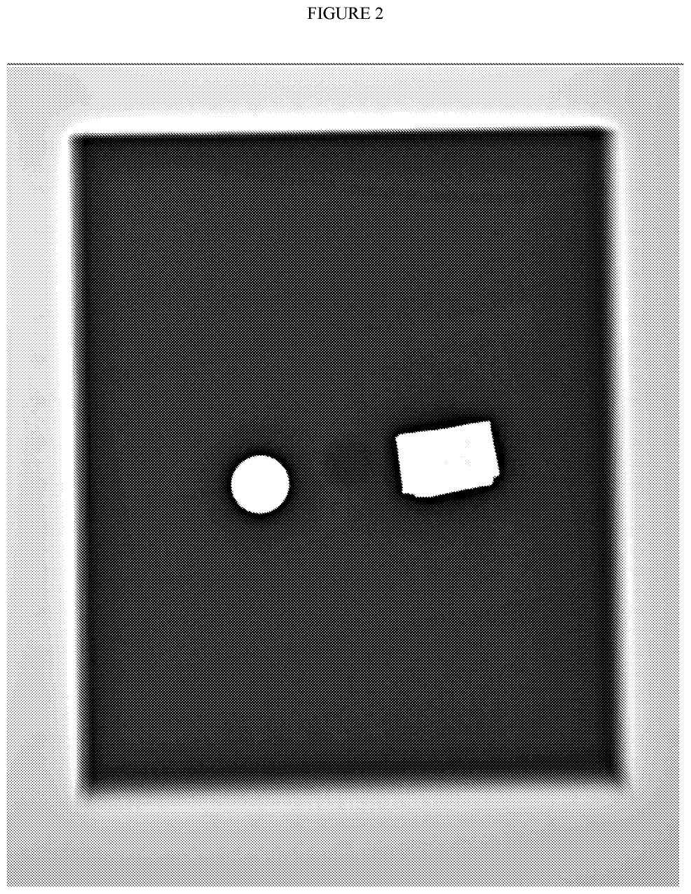

### DETAILED DESCRIPTION

As used herein the term “active pharmaceutical ingredient” (“API”) or “pharmaceutically active agent” is a drug or agent which can be employed as disclosed herein and is intended to be used in the human or animal body in order to heal, to alleviate, to prevent or to diagnose diseases, ailments, physical damage or pathological symptoms; allow the state, the condition or the functions of the body or mental states to be identified; to replace active substances produced by the human or animal body, or body fluids; to defend against, to eliminate or to render innocuous pathogens, parasites or exogenous substances or to influence the state, the condition or the functions of the body or mental states. Drugs in use can be found in reference works such as, for example, the Rote Liste or the Merck Index. Examples which may be mentioned include, for example, tretinoin.

As used herein, “pharmaceutically acceptable salts” refer to derivatives of the disclosed compounds wherein the therapeutic compound is modified by making acid or base salts thereof. Examples of pharmaceutically acceptable salts include, but are not limited to, mineral or organic acid salts of the active agent. The pharmaceutically acceptable salts include the conventional non-toxic salts, for example, from non-toxic inorganic or organic acids. For example, such conventional non-toxic salts include those derived from inorganic acids such as hydrochloric, hydrobromic, sulfuric, sulfonic, sulfamic, phosphoric, nitric and the like; and the salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and other known to those of ordinary skill in the pharmaceutical sciences. Lists of suitable salts are found in texts such as Remington's Pharmaceutical Sciences, 18th Ed. (Alfonso R. Gennaro, ed.; Mack Publishing Company, Easton, Pa., 1990); Remington: the Science and Practice of Pharmacy 19^{th} Ed. (Lippincott, Williams & Wilkins, 1995); Handbook of Pharmaceutical Excipients, 3^{th} Ed. (Arthur H. Kibbe, ed.; Amer. Pharmaceutical Assoc., 1999); the Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); and Goodman and Gilman's: the Pharmacological Basis of Therapeutics (Louis S. Goodman and Lee E. Limbird, eds.; McGraw Hill, 1992), the disclosures of which are hereby incorporated by reference.

An amount is “effective” as used herein, when the amount provides an effect in the subject. As used herein, the term “effective amount” means an amount of a compound or composition sufficient to significantly induce a positive benefit, including independently or in combinations the benefits disclosed herein, but low enough to avoid serious side effects, i.e., to provide a reasonable benefit to risk ratio, within the scope of sound judgment of the skilled artisan. For those skilled in the art, the effective amount, as well as dosage and frequency of administration, may be determined according to their knowledge and standard methodology of merely routine experimentation based on the present disclosure.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the terms “prevent,” “preventing” and “prevention” in the context of the administration of a therapy to a subject refer to the prevention or inhibition of the recurrence, onset, and/or development of a disease or condition, or a combination of therapies (e.g., a combination of prophylactic or therapeutic agents).

As used herein, the terms “therapies” and “therapy” can refer to any method(s), composition(s), and/or agent(s) that can be used in the prevention, treatment and/or management of a disease or condition, or one or more symptoms thereof.

As used herein, the terms “treat,” “treatment,” and “treating” in the context of the administration of a therapy to a subject refer to the reduction or inhibition of the progression and/or duration of a disease or condition, the reduction or amelioration of the severity of a disease or condition, and/or the amelioration of one or more symptoms thereof resulting from the administration of one or more therapies.

As used herein, the term “multi-particulates” refers to one or more unit dosage systems such as, but not limited to, pellets, beads, spheres, mini-tablets, seeds, spheroids or granules with modified drug release profile. The multi-particulates comprise a drug-release controlling and/or drug-protecting film or matrix, such as a polymeric film or matrix, whose intactness or efficiency is susceptible to certain conditions such as heat or mechanical forces that may occur during post-processing. The expression “core material” describes the nature of the interior part of multi-particulates that may also comprise a functional coat. Exemplary “core-materials” may be pellets (spherical matrix systems that contain a drug and further excipients), granules (less spherical particles that are almost entirely composed of drug) or nonpareils (spherical particles without drug).

As used herein, the term “about” when used in conjunction with a stated numerical value or range has the meaning reasonably ascribed to it by a person skilled in the art, i.e., denoting somewhat more or somewhat less than the stated value or range.

### Active Agent

The disclosure provides as an active agent a bismuth-melanin combination which absorbs x-rays (medical quality) at least as well as lead. The disclosure provides that bismuth-melanin absorbs x-rays, and other types of radiation such as Alpha radiation, beta radiation, and gamma radiation, at least as well as lead. In addition, because the specific density of the bismuth-melanin combination was about half that of an equivalent volume of lead, bismuth melanin combination weighs about half that of lead, but absorbed x-rays at least as well as lead, and thus has great utility in shielding. Furthermore, since both bismuth and melanin are essentially non-toxic (LD50 of bismuth is about 2000 mg/kg; LD50 of melanin is about >5000 mg/kg), the bismuth-melanin combination can be administered as a medicament to prevent and treat radiation poisoning.

Bismuth-Melanin (synthetic) composite, which has been produced and tested as disclosed herein absorbs X-rays at least as well as lead and is half the weight based on experiments. In addition, Bismuth-Melanin (purified Sepia) composite has been produced and also absorbs X-ray.

The invention also discloses that Bismuth-Melanin will absorb gamma rays better than lead as published by El-Bialy et al. Furthermore, Bismuth-melanin is non-toxic when ingested. For example, bismuth is present is PEPTO-BISMOL®, while melanin is the main ingredient of commercial pasta sauce for black linguini. Bismuth and Melanin are both essentially non-toxic:
LD₅₀: Bismuth>2000 mg/kg Melanin>5000 mg/kg

The structure of bismuth-melanin is as follows:

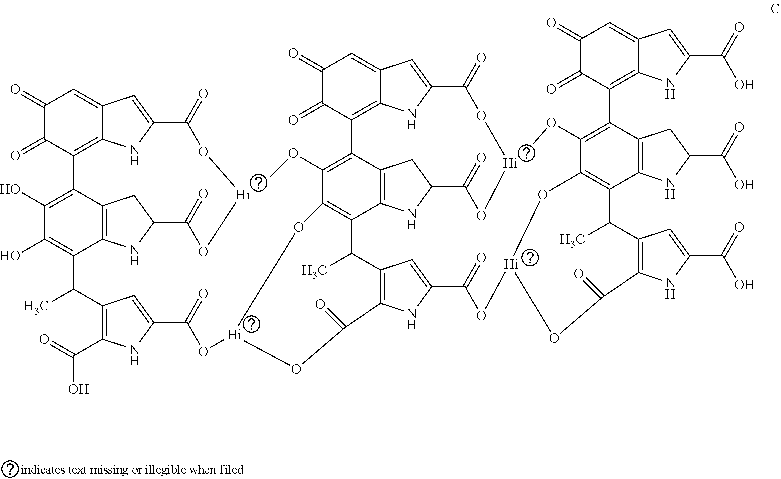

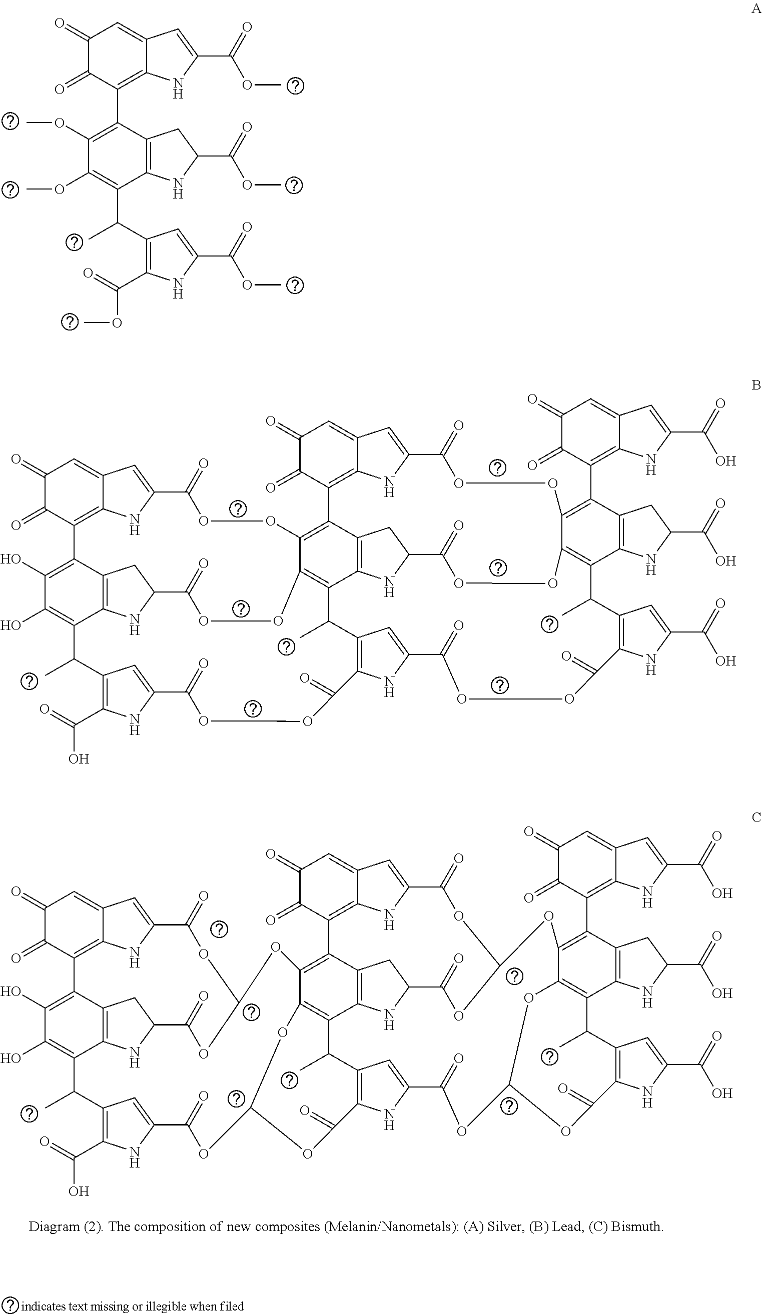

The melanins comprise a family of biopolymer pigments. A frequently used chemical description of melanin is that it is comprised of “heteropolymers of 5-6-dihydroxyindole and 5-6-dihydroxyindole-2-carboxylic acid” (Bettinger et ak, 2009). Melanins are polymers produced by polymerization of reactive intermediates. The polymerization mechanisms include, but are not limited to, autoxidation, enzyme-catalyzed polymerization and free radical initiated polymerization. The reactive intermediates are produced chemically, electrochemically, or enzymatically from precursors. Suitable enzymes include, but are not limited to, peroxidases, catalases, polyphenol oxidases, tyrosinases, tyrosine hydroxylases, and laccases. The precursors that are connected to the reactive intermediates are hydroxylated aromatic compounds. Suitable hydroxylated aromatic compounds include, but are not limited to 1) phenols, polyphenols, aminophenols and thiophenols of aromatic or polycyclicaromatic hydrocarbons, including, but not limited to, phenol, tyrosine, pyrogallol, 3-aminotyrosine, thiophenol and a-naphthol; 2) phenols, polyphenols, aminophenols, and thiophenols of aromatic heterocyclic or heteropoly cyclic hydrocarbons such as, but not limited to, 2-hydroxypyrrole, 4-hydroxy-1,2-pyrazole, 4-hydroxypyridine, 8-hydroxyquinoline, and 4,5-dihydroxybenzothiazole.

The term melanin includes naturally occurring melanin polymers as well as melanin analogs as defined below. Naturally occurring melanins include eumelanins, phaeomelanins, neuromelanins and allomelanins.

As used here, the term “melanin” refers to melanins, melanin precursors, melanin analogs, melanin variants, melanin derivatives, and melanin-like pigments, unless the context dictates otherwise. The term “melanin-like” also refers to hydrogels with melanin-like pigmentation and quinoid electrophilicity. This electrophilicity can be exploited for facile coupling with biomolecules.

As used herein, the term “melanin analog” refers to a melanin in which a structural feature that occurs in naturally-occurring or enzymatically-produced melanins is replaced by a substituent divergent from substituents traditionally present in melanin. An example of such a substituent is a selenium, such as selenocysteine, in place of sulfur.

As used herein, the term “melanin derivative” refers to any derivative of melanin which is capable of being converted to either melanin or a substance having melanin activity. An example of a melanin derivative is melanin attached to a dihydrotrigonelline carrier such as described in Bodor, N., Ann. N.Y. Acad. Sci. 507, 289 (1987), which enables the melanin to cross the blood-brain barrier. The term melanin derivatives is also intended to include chemical derivatives of melanin, such as an esterified melanin.

As used herein, the term “melanin variant” refers to various subsets of melanin substances that occur as families of related materials. Included in these subsets, but not limited thereto, are:

(1) Naturally occurring melanins produced by whole cells that vary in their chemical and physical characteristics; (2) Enzymatically produced melanins prepared from a variety of precursor substrates under diverse reaction conditions; (3) Melanin analogs in which a structural feature that occurs in (1) or (2) above is replaced by an unusual substituent divergent from the traditional; and (4) Melanin derivatives in which a substituent in a melanin produced in (1), (2) or (3) above is further altered by chemical or enzymatic means.

As used herein, the term “Melanin-like substances” refers to heteropolymers of 5-6-dihydroxyindole and 5-6-dihydroxyindole-2-carboxylic acid which have one or more properties usually associated with natural melanins, such as UV absorption or semiconductor behavior.

### Melanin Sources

### Melanin and Melanin—Like Compounds can be Obtained:

by extraction and purification from natural sources, e.g. cephalopods such as cuttlefish (e.g. Sepia) or squid (e.g. Loligo), bird feathers (e.g. from species with black strains such as Silkie chickens);
by chemical synthesis, whether water or non-water based e.g. (Deziderio, 2004) (daSilva et ak, 2004; Lawrie et ak, 2008; Pezzella et ak, 2006);
by electrochemical synthesis, e.g. (Meredith et ak, 2005);
by bioreactors created by utilization of natural or genetically altered bacteria, fungi, lichens, or viruses e.g.(della-Cioppa, 1998).

Cephalopod inks are natural composites of melanin with other materials, including peptidoglycans, amino acids, proteins, metals, and chemicals and enzymes (such as tyrosinase) which are involved in the synthesis of melanin, and other materials. Cephalopod inks include cuttlefish (such as Sepia), squid, and octopus inks. There is some variation among different species of the percentages of these components. Reports of cephalopod ink components include: Derby, C. D. 2014 Cephalopod Ink: Production, Chemistry, Functions and Applications Marine Drugs 12, 2700-2730; doi: 10.3390/mdl2052700, and Magarelli M, Passamonti P, Renieri C. 2010. Purification, characterization and analysis of sepia melanin from commercial sepia ink (Sepia Officinalis). Rev CES Med Vet Zootec; Vol 5 (2): 18-28.

### Melanin Manufacturing and Fabrication

Melanin and melanin-like compounds can be manufactured as particles, nanoparticles, dust, beads, or fibers that are woven or non-woven e.g. by methods as described by (Greiner and Wendorff, 2007), sheets e.g. (Meredith et al., 2005), films (daSilva et al., 2004), plates, bricks, chars, spheres, nodules, balls, graphite-like sheets and shards, liquids, gels, or solids (e.g. thermoplastic or thermoset), and by common chemical engineering molding and fabrication methods or custom methods. Sheets can range from one molecular layer to several millimeters. Fibers can range from nanometers to several millimeters.

The melanin material may be natural or synthetic, with natural pigments being extracted from plant and animal sources, such as squid, octopus, mushrooms, cuttlefish, and the like. In some cases, it may be desirable to genetically modify or enhance the plant or animal melanin source to increase the melanin production. Melanins are also available commercially from suppliers.

The following procedure describes an exemplary technique for the extraction of melanin from cuttlefish (Sepia Officinalis). 100 gm of crude melanin are dissected from the ink sac of 10 cuttlefish and washed with distilled water (3×100 ml). The melanin is collected after each wash by centrifugation (200×g for 30 minutes). The melanin granules are then stirred in 800 ml of 8 M Urea for 24 hours to disassemble the melanosomes. The melanin suspension is spun down at 22,000×g for 100 minutes and then washed with distilled water (5×400 ml). The pellet is washed with 50% aqueous DMF (5×400 ml) until a constant UV baseline is achieved from the washes. Finally, the pellet is washed with acetone (3×400 ml) and allowed to air dry.

Synthetic melanins may be produced by enzymatic conversion of suitable starting materials, as described in more detail hereinbelow. The melanins may be formed in situ within the porous particles or may be preformed with subsequent absorption into the porous particles. Suitable melanin precursors include but are not limited to tyrosine, 3,4-dihydroxy phenylalanine (dopa), D-dopa, catechol, 5-hydroxyindole, tyramine, dopamine, m-aminophenol, o-aminophenol, p-aminophenol, 4-aminocatechol, 2-hydroxyl-1,4-naphthaquinone (henna), 4-methyl catechol, 3,4-dihydroxybenzylamine, 3,4-dihydroxybenzoic acid, 1,2-dihydroxynaphthalene, gallic acid, resorcinol, 2-chloroaniline, p-chloroanisole, 2-amino-p-cresol, 4,5-dihydroxynaphthalene 2,7-disulfonic acid, o-cresol, m-cresol, p-cresol, and other related substances which are capable of being oxidized to tan, brown, or black melanin-like compounds capable of absorbing ultraviolet radiation when incorporated in the polymeric particle matrix of the present disclosure. Combinations of precursors can also be used.

The melanin precursor is dissolved in an aqueous solution, typically at an elevated temperature to achieve complete solution. A suitable amount of the enzyme tyrosinase (EC 1.14.18.1) is added to the solution, either before or after the melanin precursor. The concentration of tyrosinase is not critical, typically being present in the range from about 50 to about 5000 U/ml. The solution is buffered with an acetate, phosphate, or other suitable buffer, to a pH in the range from about 3 to 10, usually in the range from about 5 to 8, more usually being about 7. Melanin-like pigments can be obtained using suitable precursors even in the absence of an enzyme just by bubbling oxygen through a solution of a precursor for an adequate period of time.

Melanin material may be obtained by treatment of, e.g, cuttlefish ink or squid ink in a microwave, optionally with mixing. The inventor has found that microwaving can be used for the preparation of melanin formulations. The compositions and methods as disclosed herein may be produced and practiced using a variety of heating techniques, such as, for example, infrared heating, microwave heating, convection heating, laser heating, sonic heating, or optical heating. For example, it was found that drying melanin in a microwave oven made possible the preparation of large amount of melanin from cuttlefish ink in a very short period of time. In an exemplary embodiment, cuttlefish ink at was placed at 40° C. in a conventional oven and required 18 days to reduce the material to 40% of its original weight. In a 900 watt microwave oven, the same degree of drying was achieved in 12 minutes. The disclosure provides a method for formulation of melanin by applying a hydraulic press to melanin partially dried in a microwave oven. In exemplary embodiments, hydraulic presses for this use may range in capacity from, for example, about 1 ton/sq. in. to about 500 tons/in2 approximately. The disclosure provides a method wherein the hydraulic press applies compression of approximately 500 tons/in2. In an exemplary embodiment, commercial cuttlefish ink was dried in a 900 watt microwave oven so that the product was 30% or 35% of the initial weight. A blender was used to mix and grind the melanin. A variety of formulations were made. In one formulation, the 30% preparation was mixed with 7% iron filings, and then the blender was used to mix again. In another formulation, 35% slabs were alternated with 30% slabs to create a layered composite. Each formulation was subjected to compression in a 20 ton/in2 hydraulic press for about 20 minutes. Because the platen was approximately 3.5 in2, it is estimated that a force of approximately 3265 pounds/sq. in. was exerted on each sample formulation.

In exemplary embodiments, formulations as disclosed herein may comprise active agent at a concentration of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80%, In exemplary embodiments, formulations as disclosed herein may comprise active agent at a concentration of about 1 to about 20%, of about 5% to about 25%, about 10% to about 20%, or about 15% to about 18%, about 30% to about 70%, about 35% to about 65%, about 63.13%, and about 40% to about 64% w/w.

In an exemplary formulation as disclosed herein, the active agent will represent approximately 1 wt % to 75 wt %, preferably 2 wt % to 30 wt %, more preferably 5 wt. % to 20 wt. % of the total weight.

In other embodiments, the pharmaceutical compositions further comprise one or more additional materials such as a pharmaceutically compatible carrier, binder, viscosity modifier, filling agent, suspending agent, flavoring agent, sweetening agent, disintegrating agent, surfactant, preservative, lubricant, colorant, diluent, solubilizer, moistening agent, stabilizer, wetting agent, anti-adherent, anti-foaming agent, antioxidant, chelating agent, antifungal agent, antibacterial agent, or one or more combination thereof.

Protection from Radiation

It has been reported that melanin absorbs beta particles, gamma rays, X-rays, infrared, visible, ultraviolet, the remainder of the electromagnetic spectrum, and combinations thereof. The present disclosure includes the discovery that melanin can be used alone, or in composites with other materials such as lead and polymers, to absorb and prevent destruction by radiation, e.g., for shielding, armor, and aerospace applications such as airplane and space vehicle construction parts.

The present disclosure includes the discovery that bismuth-melanin composite can be used alone, or in composites with other materials for:
a. shielding of radiation from sources like uranium and radium.
b. to degrade, encapsulate and shield from living and non-living radioactive particles in sizes from nanometers to millimeters.
c. to shield personnel and equipment from radiation from depleted uranium used in weaponry or armor.

The present disclosure includes the discovery that bismuth-melanin can be used alone, or in composites with other materials not only by covering a human or other organism by bismuth-melanin, alone or in mixture with other materials: It can be accomplished by ingestion, injection, or other internal administration of these compounds or composites.

Furthermore, the bismuth-melanin, can be used to mitigate the destructive biological effects of radiation, even if the radiation has been absorbed. For instance, radiation creates free radicals in biological tissues which creates great damage in the hematopoietic and gastrointestinal systems. bismuth-melanin is known to absorb such free radicals and mitigate such damage.

### Therapy

The active agents as disclosed herein may be administered to a subject concurrently, the term “concurrently” is not limited to the administration of the cancer therapeutics at exactly the same time, but rather, it is meant that they are administered to a subject in a sequence and within a time interval such that they can act together (e.g., synergistically to provide an increased benefit than if they were administered otherwise). For example, a first prophylactically and/or therapeutically effective regimen can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the second cancer therapeutic, to a subject in need thereof. In various embodiments, the cancer therapeutics are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the cancer therapeutics are administered within the same office visit. In another embodiment, the combination cancer therapeutics are administered at 1 minute to 24 hours apart.

### Dosage Forms

The compositions comprising active agents as disclosed herein can provided in the form of a minicapsule, a capsule, a tablet, an implant, a troche, a lozenge (minitablet), a temporary or permanent suspension, an ovule, a suppository, a wafer, a chewable tablet, a quick or fast dissolving tablet, an effervescent tablet, a granule, a film, a sprinkle, a pellet, a bead, a pill, a powder, a triturate, a platelet, a strip or a sachet. Compositions can also be administered after being mixed with, for example yoghurt or fruit juice and swallowed or followed with a drink or beverage. These forms are well known in the art and are packaged appropriately. The compositions can be formulated for oral or rectal delivery.

Tablets prepared for oral administration according to the invention, and manufactured using direct compression, will generally contain other inactive additives such as binders, lubricants, disintegrants, fillers, stabilizers, surfactants, coloring agents, and the like. Binders are used to impart cohesive qualities to a tablet, and thus ensure that the tablet remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Lubricants are used to facilitate tablet manufacture, promoting powder flow and preventing particle capping (i.e., particle breakage) when pressure is relieved. Useful lubricants are magnesium stearate 0, calcium stearate, stearic acid, and hydrogenated vegetable oil (preferably comprised of hydrogenated and refined triglycerides of stearic and palmitic acids at about 1 wt. % to 5 wt. %, most preferably less than about 2 wt. %). Lubricants may be present in a concentration of, for example, from about 0.25 wt. % to about 3 wt. %, 0.5 wt. % to about 2.0 wt. %, from about 0.75% to about 1.5%..

Disintegrants are used to facilitate disintegration of the tablet, thereby increasing the erosion rate relative to the dissolution rate, and are generally starches, clays, celluloses, algins, gums, or crosslinked polymers (e.g., crosslinked polyvinyl pyrrolidone). Fillers include, for example, materials such as silicon dioxide, titanium dioxide, alumina, talc, kaolin, powdered cellulose, and microcrystalline cellulose, as well as soluble materials such as mannitol, urea, sucrose, lactose, lactose monohydrate, dextrose, sodium chloride, and sorbitol. Solubility-enhancers, including solubilizers per se, emulsifiers, and complexing agents (e.g., cyclodextrins), may also be advantageously included in the present formulations. Stabilizers, as well known in the art, are used to inhibit or retard drug decomposition reactions that include, by way of example, oxidative reactions. Disintegrants may be present in a concentration of, for example, from about 0.25 wt. % to about 3 wt. %, 0.5 wt. % to about 2.0 wt. %, from about 0.75% to about 1.5%.

Shellac, also called purified lac, a refined product obtained from the, resinous secretion of an insect. This coating dissolves in media of pH>7.

Colorants, detackifiers, surfactants, antifoaming agents, lubricants, stabilizers such as hydroxy propyl cellulose, acid/base may be added to the coatings besides plasticizers to solubilize or disperse the coating material, and to improve coating performance and the coated product.

In carrying out the method as disclosed herein, the combination of the invention may be administered to mammalian species, such as dogs, cats, humans, etc. and as such may be incorporated in a conventional systemic dosage form, such as a tablet, capsule, or elixir. The above dosage forms will also include the necessary carrier material, excipient, viscosity modifier, lubricant, buffer, antibacterial, bulking agent (such as mannitol), anti-oxidants (ascorbic acid of sodium bisulfate) or the like.

The dose administered may be carefully adjusted according to age, weight and condition of the patient, as well as the route of administration, dosage form and regimen and the desired result.

The compositions of the invention may be administered in the dosage forms in single or divided doses of one to four times daily, or may be administered multiple times per day. It may be advisable to start a patient on a low dose combination and work up gradually to a high dose combination.

Tablets of various sizes can be prepared, e.g., of about 2 to 2000 mg in total weight, containing one or both of the active ingredients, with the remainder being a physiologically acceptable carrier of other materials according to accepted practice. Gelatin capsules can be similarly formulated.

Liquid formulations can also be prepared by dissolving or suspending one or the combination of active substances in a conventional liquid vehicle acceptable for administration so as to provide the desired dosage in, for example, one to four teaspoonfuls.

Dosage forms can be administered to the patient on a regimen of, for example, one, two, three, four, five, six, or other multiple doses per day.

In order to more finely regulate the dosage schedule, the active substances may be administered separately in individual dosage units at the same time or carefully coordinated times. The respective substances can be individually formulated in separate unit dosage forms in a manner similar to that described above.

In formulating the compositions, the active substances, in the amounts described above, may be compounded according to accepted practice with a physiologically acceptable vehicle, carrier, excipient, binder, viscosity modifier, preservative, stabilizer, flavor, etc., in the particular type of unit dosage form.

Examples of lipids that may be employed in the compositions and methods as disclosed herein include, but are not limited to, fats, oils, waxes, fatty acids, fatty acid esters, glycerides, fatty alcohols, hydrogenated vegetable oil, soybean oil, phospholipids, terpenes and the like or combinations thereof. Suitable waxes that may be employed include, but are not limited to, natural waxes, such as animal waxes, vegetable waxes, and petroleum waxes (i.e., paraffin waxes, microcrystalline waxes, petrolatum waxes, mineral waxes), and synthetic waxes. Non-limiting examples include, but are not limited to, spermaceti wax, carnauba wax, Japan wax, bayberry wax, flax wax, beeswax, Chinese wax, shellac wax, lanolin wax, sugarcane wax, candelilla wax, paraffin wax, microcrystalline wax, petrolatum wax, carbowax, and the like, or mixtures thereof. Mixtures of these waxes with the fatty acids may also be used. Non-limiting examples of oils that may be employed include, castor oil, soybean oil, and the like or combinations thereof. Fatty acids that may be employed in the present invention include, but are not limited to, decenoic acid, docosanoic acid, stearic acid, palmitic acid, lauric acid, myristic acid, and the like, and mixtures thereof. Suitable fatty alcohols that may be employed in the compositions as disclosed herein include, but are not limited to, cetyl alcohol, stearyl alcohol or mixtures thereof. Suitable hydrogenated vegetable oils that may be employed in the compositions as disclosed herein, include but are not limited to, hydrogenated palm kernel oil, hydrogenated peanut oil, hydrogenated palm oil, hydrogenated rapeseed oil, hydrogenated rice bran oil, hydrogenated soybean oil, hydrogenated sunflower oil, hydrogenated castor oil, hydrogenated cottonseed oil, and the like, and mixtures thereof. In one embodiment, lipids may be employed in case the carrier particles being prepared are solid lipid nanoparticles, lipid-based nanoparticles or microparticles, nanoemulsions, microemulsions, liposomes, and the like or combinations thereof.

### Packaging/Treatment Kits

The disclosure provides a kit for conveniently and effectively carrying out the methods in accordance with the present disclosure. Such kits may be suited for the delivery of solid oral forms such as tablets or capsules. Such a kit may include a number of unit dosages. Such kits can include a means for containing the dosages oriented in the order of their intended use. An example of a means for containing the dosages in the order of their intended uses is a card. An example of such a kit is a “blister pack”. Blister packs are well known in the packaging industry and are widely used for packaging unit dosage forms. If desired, the blister can be in the form of a childproof blister, i.e. a blister that is difficult for a child to open, yet can be readily opened by an adult. If desired, a memory aid can be provided, for example in the form of numbers, letters, or other markings or with a calendar feature and/or calendar insert, designating the days and the sections of a day in the treatment schedule in which the dosages can be administered, such as, for example, an AM dose is packaged with a “midday” and a PM dose.; or an AM dose is packaged with a PM dose. Alternatively, placebo dosages, or vitamin or dietary supplements, either in a form similar to or distinct from the active dosages, can be included.

The disclosure provides compositions, including preparations, formulations and/or kits, comprising combinations of ingredients, as described above (including the multi-ingredient combinations of drugs of the invention), that are serviceable as therapies for treating, preventing or improving conditions, states and disease as provided in the invention. In one aspect, each member of the combination of ingredients is manufactured in a separate package, kit or container; or, all or a subset of the combinations of ingredients are manufactured in a separate package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package consists two or more separate compartments. This blister package is made up of two separate material elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the agents of the invention. In one aspect, a blister pack of the invention comprises a moulded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack.

In one aspect, a blister pack also comprises a method of packaging where the compositions comprising combinations of ingredients of the invention are contained in-between a card and clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. of the invention are used, and they can comprise of two vacuum-formed PVC sheets meshed together at the edges, with the informative card inside.

In one aspect, blister packaging comprises at least two components (e.g., is a multi-ingredient combination of drugs of the invention): a thermoformed “blister” which houses the product (e.g., a combination of the invention), and then a “blister card” that is a printed card with an adhesive coating on the front surface. During the assembly process, the blister component, which is most commonly made out of PVC, is attached to the blister card using a blister machine. Conventional blister packs can also be sealed.

As discussed herein, the products of manufacture of the invention can comprise the packaging of the therapeutic drug combinations of the invention, alone or in combination, as “blister packages” or as a plurality of packettes, including as lidded blister packages, lidded blister or blister card or packets, or a shrink wrap.

In one aspect, any of the invention's products of manufacture, including kits or blister packs, include memory aids to help remind patients when and how to take the agents of the invention.

The treatment kits can be constructed in a variety of forms familiar to one of ordinary skill in the art. The kits comprise at least one unit dosage of an active for administration according to a daily regimen and a means for containing the unit dosages. The treatment kits can, for example, be constructed for administration once daily, twice daily, thrice daily, four times daily, multiple administrations daily, or other dosage regimens. The kits comprise a means for the daily administration of an agent of the invention. In one embodiment the kits include from about one to about four unit dosages.

In one embodiment, the means for containing the unit dosages is a card, including, for example, a card that is capable of being folded. This card will be referred to herein as a main card, or alternatively a principal card or a first card, to distinguish it from additional optional cards, circulars, or other such materials which can be associated with the kit. This main card can be folded with a simple crease, or alternatively, with a double crease, so as to exhibit a spine, similar to the spine of a closed book. The main card can comprise a printable surface, i.e. a surface upon which the product name, appropriate administration instructions, product information, drawings, logos, memory aids, calendar features, etc. can be printed. The main card can comprise a means for containing said unit dosage or different dosages designated for different time of the day, and a memory aid for administering said unit dosage or dosages. The main card, especially if it is prepared from two or more laminated paperboard surfaces, can comprise a slit or pocket, for example in one of the inner paperboard surfaces of the folded card. The slit or pocket can be used to contain a removable secondary card, i.e., a second card or insert card, which is not permanently attached or affixed to the main card.

The memory aid can include a listing of the days of the week, i.e., Sunday, Monday, Tuesday, Wednesday, Thursday, Friday, and Saturday, with appropriate spaces for the patient to select and indicate on the card the preferred day of the week on which to administer the therapy. The memory aid can include a listing of the time of day with appropriate spaces for the patient to select and indicate on the card the preferred time of day (e.g.: AM, PM, midday) at which to administer the therapy. The memory aid can also include removable stickers having an appropriate pressure sensitive adhesive to facilitate easy removal and refastening to a desired surface such as a calendar or dayminder. The removable stickers can be located on the main card, or can be located on the secondary card which is constructed so that it can be readily inserted into and removed from the optional slit in the main card. Additionally, the optional slit can contain additional patient information and other circulars.

Other means for containing said unit dosages can include bottles and vials, wherein the bottle or vial comprises a memory aid, such as a printed label for administering said unit dosage or dosages. The label can also contain removable reminder stickers for placement on a calendar or dayminder to further help the patient to remember when to take a dosage or when a dosage has been taken.

The invention will be illustrated in more detail with reference to the following Examples, but it should be understood that the present invention is not deemed to be limited thereto.

### EXAMPLES

### Example 1

An experiment was conducted to determine the x-ray absorption of a bismuth-melanin sample.

The sample was manufactured about 4/9/21 using the following procedure.

### A. Materials and Equipment:

Bismuth oxide from Sigma Chemical
Melanin (synthetic) from Sigma Chemical
NaOH
Water bath
Mortar for grinding
Manual Tableting press (single tablet)

### B. Preparation Procedure:

15 g of Bismuth oxide was mixed with 1 g of melanin and dissolved in 20 ml of 1N NaOH in a 100-ml beaker.
This mixture was then placed in a water bath at 100° c. for about an hour till the excess liquid is evaporated.
After it is heated for an hour, the mixture is left for cooling at room temperature where it dries up.
This dried mixture was transferred into a mortar and grinded to form a fine powder.
This powder was transferred into a tableting press (pressure of 360 bar=5221 pounds per square inch) where it is compressed in the form of a circular disc.

### C. The Bismuth-Melanin Disk Produced had the Following Characteristics

Color: Brown
Diameter: 20.04 mm
Thickness: 6.54 mm
Density: 5.21 g/cc
Weight: 10.87 g
   The x-Ray Absorption of the Sample was Tested as Follows.
   A Lead Rectangle Control was Produced with the Following Characteristics:
Thickness: 6.29 mm
Width: 21.66 mm
Length: 34.03 mm
Density: approx. 11.4 gm/cc

Thus, the thickness of the bismuth-melanin disk was about the same as that of the lead rectangle control

A medical x-ray machine was set at the following parameters:
15 mA, 50 KVP, 0.2 sec

The sample disk was put adjacent to the rectangular lead control, and an X-ray was taken.

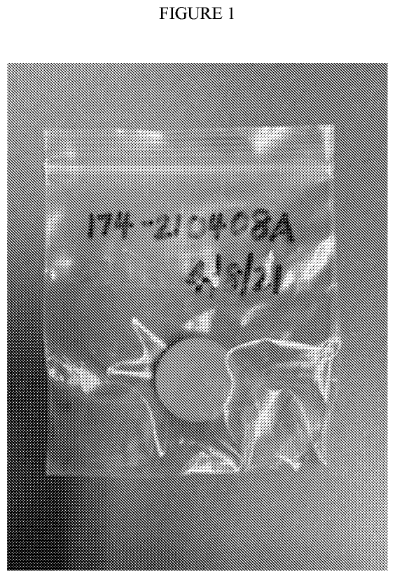

The resulting x-ray radiograph is shown in FIG. 1.

### CONCLUSIONS

1. Because the sample disk is at least as white as the lead rectangle, it was concluded that bismuth-melanin sample absorbed x-rays at least as well as lead.
2. Because the specific density of the bismuth-melanin combination was about half that of an equivalent volume of lead, it was concluded that the bismuth melanin combination weighed about half that of lead, but absorbed x-rays at least as well as lead, which would have great utility in shielding.
3. It is also noted that since both bismuth and melanin are essentially non-toxic (LD50 of bismuth is about 2000 mg/kg; LD50 of melanin is about >5000 mg/kg), the bismuth-melanin combination could be administered as a medicament to prevent and/or treat radiation poisoning.

While the invention has been described in detail and with reference to specific examples thereof, it will be apparent to one skilled in the art that various changes and modifications can be made therein without departing from the spirit and scope thereof.

What is claimed is:

1. A pharmaceutical composition comprising:
a therapeutically effective amount of a bismuth-melanin composite, pharmaceutically acceptable salts thereof, and combinations thereof, and
at least one pharmaceutically acceptable excipient.

2. A method for the treatment and/or prevention of acute radiation syndrome (ARS) in a subject in need thereof comprising: administering the composition of claim 1 to the subject, wherein the administration of the composition treats and/or prevents ARS in the subject.

3. The method of claim 2, wherein the radiation is selected from the group consisting of beta particles, gamma rays, X-rays, infrared, visible, ultraviolet, the remainder of the electromagnetic spectrum, and combinations thereof.

4. A kit for the treatment, amelioration or prevention of a condition selected from the group consisting of acute radiation syndrome (ARS), in a patient in need thereof comprising:
(a) the pharmaceutical composition of claim 1; and
(b) at least one blister package; a lidded blister; a blister card or packet; a clamshell; an intravenous (IV) package, IV packette or IV container; a bottle; a metal tube; a laminate tube; a plastic tube; a dispenser; a pressurized container; a barrier container; a package; a tray or a shrink wrap, comprising the pharmaceutical composition of (a) and instructions for use of the pharmaceutical composition.

5. A product of manufacture comprising a blister package; a lidded blister; a blister card or packet; a clamshell; an intravenous (IV) package, IV packette or IV container; a bottle; a metal tube; a laminate tube; a plastic tube; a dispenser; a pressurized container; a barrier container; a package; a tray or a shrink wrap comprising the pharmaceutical composition of claim 1 and instructions for use of the composition.

6. A radiation protection material formed using a composition comprising a bismuth-melanin composite; and
Optionally additional materials.

7. The radiation protection material of claim 6, wherein the radiation protection material is manufactured in a form selected from the group consisting of particles, nanoparticles, dust, beads, fibers that are woven, fibers that are non-woven, sheets, films, slabs, plates, bricks, chars, spheres, nodules, balls, graphite-like sheets and shards, liquids, gels, solids, thermoplastic solids, and thermoset solids.

8. A process for forming a radiation protection material comprising:
mixing a bismuth-melanin composite; and
at least one additional non-melanin material; and
shaping the resultant material into an article.

9. The process of claim 8, wherein the radiation protection material is manufactured in a form selected from the group consisting of particles, nanoparticles, dust, beads, fibers that are woven, fibers that are non-woven, sheets, films, plates, bricks, chars, spheres, nodules, balls, graphite like sheets and shards, liquids, gels, solids, thermoplastic solids, and thermoset solids.

10. The process of claim 9 wherein the radiation protection material absorbs radiation selected from the group consisting of beta particles, gamma rays, X-rays, infrared, visible, ultraviolet, the remainder of the electromagnetic spectrum, and combinations thereof.
